(19) United States
(12) Patent Application Publication
(10) Pub. No.: US 2022/0257830 A1
(43) Pub. Date: Aug. 18, 2022

(54) RIGID RESORBABLE MATERIALS WITH POLYMER AND ORGANIC FILLERS

(71) Applicant: Evonik Operations GmbH, Essen (DE)

(72) Inventors: Jian-Feng Zhang, Vestavia, AL (US); Teng Xue, Tianjin (CN); Marshall Scott Jones, Bessemer, AL (US); Balaji Prabhu, Hoover, AL (US); Andreas Karau, Gelnhausen (DE)

(73) Assignee: Evonik Operations GmbH, Essen (DE)

(21) Appl. No.: 17/627,800

(22) PCT Filed: Jul. 14, 2020
(86) PCT No.: PCT/EP2020/069864

§ 371 Date: Jan. 17, 2022

Related U.S. Application Data
(63) Continuation of application No. 16/517,124, filed on Jul. 19, 2019.

(51) Int. Cl. A61L 27/26; B33Y 10/00; B33Y 70/00; B33Y 80/00; B29C 64/118; B29C 64/153; B29C 64/165; B29C 48/00; A61K 47/34; A61K 47/36; A61L 27/54; B29C 45/00; B29C 51/00; C08L 1/02; C08L 3/02

(52) U.S. Cl. CPC A61L 27/26; B29K 2067/04; B33Y 70/00; B33Y 80/00; B29C 64/118; B29C 64/153; B29C 64/165; B29C 48/022; A61K 47/34; A61K 47/36; A61L 27/54; B29C 45/0001; B29C 51/002; C08L 1/02; C08L 3/02; A61L 2430/02; B33Y 10/00

(57) ABSTRACT

This invention relates to the composition of flexible resorbable polymers with rigid resorbable fillers. The invention further relates to processing of flexible resorbable polymers with rigid resorbable fillers. The invention relates also to the use of such materials for applications in fast degradation applications. The invention also relates to the composition of flexible resorbable polymers with rigid resorbable for making shape memory materials. This invention also related to the processing of such materials by extrusion, injection molding, thermoforming, solvent mixing, and additive manufacturing. The invention also relates to the use of such materials as bone filler, vascular closure and other hemostasis devices, aneurysms, and stent applications. The invention also relates to the use of such materials as drug delivery platforms.

### FIELD OF THE INVENTION

This invention relates to the composition of flexible resorbable polymers with rigid resorbable fillers. The invention further relates to processing of flexible resorbable polymers with rigid resorbable fillers. The invention also relates to the use of such materials for applications in fast degradation applications. The invention also relates to the composition of flexible resorbable polymers with rigid resorbable for making shape memory materials. This invention also related to the processing of such materials by extrusion, injection molding, thermoforming, solvent mixing, and additive manufacturing. The invention also relates to the use of such materials as bone filler, vascular closure and other hemostasis devices, aneurysms, and stent applications. The invention also relates to the use of such materials as drug delivery and drug release platforms.

### BACKGROUND OF THE INVENTION

In implantable medical devices, such as ligating clips, cardiovascular stents, closure devices, there is a need for rigid resorbable polymers that can resist deformation under load or pressure and degrade in less than one year. Among synthetic resorbable polymers, poly(L-lactide) (PLLA), poly(D,L-lactide) (PDLA), poly(lactide-co-glycolide) (PLGA), poly(L-lactide-co-D,L-lactide) (PLDLLA) represent a category of strong, rigid but brittle polymers with tensile strength in a range of 50-70 MPa, elastic modulus (a measure of stiffness/rigidity) between 2-4 GPa and elongation at break less than 5%. Furthermore, these materials take more than 1 year to degrade. Poly(caprolactone) and its copolymer with L-lactide represent flexible polymers with even longer degradation time. Polyglycolide (PGA) degrades in about 4-6 months. PGA tends to be a rigid and brittle material, only multifilament braided or very fine monofilament are suitable for suture use. Polydioxanone (PDO) has been characterized as flexible (i.e., low modulus) biodegradable polymer with low crystallization rate and crystallinity. The flexibility has merited PDO as surgical suture.

U.S. Pat. No. 5,997,568 included 0.01-1 percent by weight with about 0.05 to about 0.5 percent by weight being preferred of resorbable particles with size in the range of 0.1-1 μm as nucleation agent for PDO forming suture. U.S. Pat. No. 4,591,630 claimed thermal annealing of neat PDO to improve mechanical strength for ligating clip application. Inorganic fillers including calcium carbonate (CaCO₃), montmorillonite, organically modified clay, hydroxyapatite or boron nitride, and sepiolite, have been introduced to PDO to improve mechanical properties, fast degradation, and thermal stability (Y. Bai, P. Wang, Z. Fan, et al. Effect of particle size and surface modification on mechanical properties of poly(para-dioxanone)/inorganic particles. Polym. Compos., 2012, 33: 1700-1706. DOI 10.1002/pc.22303; F. Y Huang, Y. Z. Wang, et al. Preparation and characterization of a novel biodegradable poly(p-dioxanone)/montmorillonite nanocomposite. J. Polym. Sci. part A. Polym. Chem., 2005, 43: 2298-2303; M. Zubitur, A. Fernandez, A. Mugica, M. Cortaza. Novel nanocomposites based on poly(p-dioxanone) and organically modified clays. Phys. Status Solidi A, 2008, 205: 1515-1520; M. A. Sabino, L. Sabater, G. Ronca, A. J. Mueller. The effect of hydrolytic degradation on the tensile properties of neat and reinforced poly(p-dioxanone). Polym. Bulletin. 2002, 48: 291-298; Z. C. Qiu, J. J. Zhang, et al. preparation of poly(p-dioxanone)/sepiolite nanocomposites with excellent strength/toughness balance via surface-initiated polymerization. Ind. Eng. Chem. Res., 2011, 50: 10006-10016. doi.org/10.1021/ie200106f). These reinforced PDO except by hydroxyapatite or boron nitride, have not been reported for medical applications. Rigid resorbable organic fillers, in particular in the form of particles, have not been reported for mechanical strength improvement of flexible polymeric matrix. It is clear that there is a gap for a rigid and tough resorbable polymer suitable for medical applications.

### SUMMARY OF THE INVENTION

The objective of this present invention is to provide rigid and tough resorbable materials that can be used as bone filler, vascular closure, stents, shape memory, fast degradation, drug delivery and drug release applications from flexible polymeric raw materials.

Another objective of this present invention is to provide such materials that can be processed by extrusion, injection molding, thermoforming, additive manufacturing, and by solvent mixing.

The present invention is directed to a flexible resorbable polymer.

In another aspect, disclosed are rigid resorbable fillers.

In still another aspect, disclosed is a composition comprising a flexible resorbable polymer and resorbable filler(s).

In still another aspect, disclosed are resorbable fillers that are more rigid than the resorbable polymers.

In still another aspect, disclosed are rigid resorbable fillers and flexible resorbable polymers have different melting temperatures.

In still another aspect, disclosed is a composition comprising a flexible resorbable polymer and rigid resorbable fillers.

In still another aspect, disclosed is a composition comprising: a flexible resorbable polymer, another flexible resorbable polymer, and rigid resorbable fillers.

In still another aspect, disclosed is a composition comprising a flexible resorbable polymer, rigid resorbable fillers, and an active pharmaceutical ingredient.

In still another aspect, disclosed rigid resorbable fillers comprising an active pharmaceutical ingredient.

In still another aspect, disclosed is a process for preparing a composition of flexible resorbable polymer, and rigid resorbable fillers by thermal processing steps of extrusion and injection molding to form resorbable material composition.

In still another aspect, disclosed is thermal processing flexible resorbable polymer, rigid resorbable fillers, and active pharmaceutical ingredients.

In still another aspect, disclosed is a thermal process where temperature is above the melting temperature of a flexible resorbable polymer and below the melting temperature of rigid resorbable fillers.

In still another aspect, disclosed is a thermal process where temperature is above the melting temperature of a flexible resorbable polymer and below the temperature which can adversely affect properties of active pharmaceutical ingredients.

In still another aspect, disclosed is a process for preparing a composition of flexible resorbable polymer, and rigid resorbable fillers by solvent mixing.

In still another aspect, disclosed is a process for preparing a composition of flexible resorbable polymer, rigid resorbable fillers, and active pharmaceutical ingredients by solvent mixing.

In still another aspect, disclosed is a solvent can dissolve the flexible resorbable polymer, but cannot dissolve the rigid resorbable fillers.

In still another aspect, disclosed is a solvent can dissolve the flexible resorbable polymer, but cannot dissolve the rigid resorbable fillers with active pharmaceutical ingredients.

In still another aspect, disclosed is a material has shape memory effect.

Additional advantages will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION OF THE INVENTION

Before the present materials and processes are disclosed and described, it is to be understood that the aspects described herein are not limited to specific processes, polymers, synthetic methods, articles, devices, or uses as such can, of course, vary. It is also to be understood that the terminology used herein is for the purpose of describing particular aspects only and, unless specifically defined herein, is not intended to be limiting.

Disclosed herein are flexible resorbable polymers and rigid resorbable fillers. The disclosed resorbable polymers provide the advantage of a biodegradable profile, and the process ability with rigid filler(s) to form composites or blends having better mechanical properties than individual constituents. Another advantage is that it can be extended to a vast range of applications, including shape memory, bone filler, vascular closure, stent, fast degradation applications, 3D printing, and drug delivery platforms etc.

### Definition of Terms

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “wt. %” means weight percent.

The term “w/w” means weight per weight.

The term “flexible” refers to polymers that are able to bend or deform without breaking.

The term “rigid” or “rigidity” refers to a property of a polymer that is described by modulus. It is a measure of a polymer resistance to bend or deform when a force is applied to the polymer.

The term “amorphous” refers to polymers that have no detectable crystal structure. The polymer chains are disorganized. A skilled person in the field of polymers knows how to determine amorphous polymers, for example by differential scanning calorimetry or X-ray.

The term “semi-crystalline” refers to polymers exhibiting organized and tightly packed molecular chains with sharp melt points. Such polymers remain solid until a given quantity of heat is absorbed and then rapidly change into flowable liquid. A skilled person in the field of polymers knows how to determine semi-crystalline polymers, for example by differential scanning calorimetry or X-ray.

For the purposes of the present invention, the term “shape memory effect” refers to polymers that have the ability to return from a deformed state (temporary shape) to their original (permanent) shape induced by an external stimulus (trigger), such as temperature change.

For the purposes of the present invention, the term “resorbable” or “biodegradable” refers to polymers that dissolve or degrade in vivo within a period of time that is acceptable in a particular therapeutic situation. Such dissolved or degraded product may include a smaller chemical species. Degradation can result, for example, by enzymatic, chemical and/or physical processes. Biodegradation takes typically less than five years and usually less than one year after exposure to a physiological pH and temperature, such as a pH ranging from 6 to 9 and a temperature ranging from 22° C. to 40° C.

For the purposes of the present invention, the term “3D printed part” refers to a part printed by a 3D printer. A 3D printer includes, but are not limited to, bioplotter, fused filament fabrication (FFF), selective laser sintering (SLS), and stereolithography (SLA). A 3D printed part can also be a bioprinted part.

For the purposes of the present invention, the term “thermal printer” refers to a printer that can print thermoplastics. A thermal printer includes, but are not limited to, FFF, and binder jetting.

Suitable flexible resorbable polymers of the invention include without limitation a polydioxanone, a polycaprolactone, a copolymer of polydioxanone-polycaprolactone, a copolymer of poly(lactide-co-trimethylene carbonate), a copolymer of poly(glycolide-co-trimethylene carbonate), a copolymer of poly(lactide-co-caprolactone), a poly(orthoester), a poly(phosphazene), a poly(hydroxybutyrate), a copolymer containing poly(hydroxybutarate), a biodegradable polyurethane, a poly(amino acid), a polyetherester, polyphosphoesters, a polyethylene glycol (PEG), a copolymer of polylactide-co-PEG, a copolymer of PGA-co-PEG, a copolymer of PCL-co-PEG, a copolymer of PDO-co-PEG, a polyanhydride, a copolymer of PEG and a polyorthoester, a copolymer of polyhydrobutyrate-co-polyhydroxyvalerate, and copolymers, terpolymers, or a mixture thereof;

Suitable rigid resorbable fillers include but are not limited to poly(lactide), a poly(glycolide), a copolymer of poly(L-lactide-co-D,L-lactide) (PLDLLA), a poly(lactide-co-glycolide), a polyesteramide, a polylactide sterocomplex, starch granules, cellulose microcrystals, chitin whisker, or a mixture thereof.

Suitable rigid resorbable natural fillers include but are not limited to starch granules, cellulose microcrystals, chitin whisker, collagen, cross-linked collagen, silk, or a combination thereof.

Suitable rigid resorbable synthetic fillers include but are not limited to poly(lactide), a poly(glycolide), a copolymer of poly(L-lactide-co-D,L-lactide) (PLDLLA), a poly(lactide-co-glycolide), a polyesteramide, a polylactide sterocomplex.

Suitable rigid resorbable filler forms include but are not limited to powders, fines, granules, spheres, particles, crystalline whiskers, or a mixture thereof.

The flexible resorbable polymer or the rigid reasorbable particles can comprise one or more residues of lactic acid, glycolic acid, lactide, glycolide, caprolactone, hydroxybutyrate, hydroxyvalerates, dioxanones, polyethylene glycol, polyethylene oxide, or a combination thereof. In some aspects, the resorbable polymer comprises one or more lactide residues. The polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers.

When poly(lactide-co-glycolide), poly(lactide), or poly(glycolide) is used, the amount of lactide and glycolide in the polymer can vary. For example, the biodegradable polymer can contain 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the resorbable polymer can be poly(lactide), 95:5 poly(lactide-co-glycolide) 85:15 poly(lactide-co-glycolide), 75:25 poly(lactide-co-glycolide), 65:35 poly(lactide-co-glycolide).

The solvent used in the present invention include, but are not limited to acetone, chloroform, dichloromethane, acetonitrile, 1,4-dixone, di methylsulfoxide, dimethyl formamide, hexafluoroisopropanol (HFIP), polyethylene glycol, or N-Methyl-2-Pyrrolidone (NMP).

The non-solvent used in the present invention include, but are not limited to ethanol, methanol, water, cyclohexane, hexane, pentane, hydrogen peroxide, diethyl ether, tert-butyl methyl ether (TBME), phosphate buffer saline solution (PBS), or a mixture thereof.

In another embodiment, active pharmaceutical ingredient include, but are not limited to Alendronate, Acetaminophen, Olpadronate, Etidronate, Colecalciferol (vitamin D), Tocopherol (vitamin E), Pyridoxin (vitamin B6), Cobalamine (vitamin B12) Platelet-derived growth factor (PDGF), Glycine, Lysine, penicillin, cephalosporin, lamivudine, tetracycline, and zidovudine.

In another embodiment, thermal processing incudes, but are not limited to, single screw extrusion, twin screw extrusion, compression molding, thermoforming, additive manufacturing or 3D printing, and injection molding.

The typical degradation profiles of flexible resorbable polymers, rigid resorbable particles, or the materials comprising flexible resorbable polymers and rigid resorbable particles thereof can be at least two weeks, at least one month, at least 3 months, at least 6 months, at least 9 months, at least 12 months, at least 18 months, or at least 24 months.

In one embodiment, shape memory effect is achieved by the following process. The polymer pellets are heated above melting temperature of the hard segment. Then the polymer is melted and allows for thermal processing. The polymer is cooled and able to hold a predetermined shape. The predetermined shape is fixed, but the polymer can return to its primary shape if it is exposed to a temperature change. The polymer is heated to the transition temperature (denoted as Ttrans), together with the application of an external force, the polymer is left in a temporary shape. The external force is still applied on the polymer, but the temperature is lowered until it reaches a temporary shape. Once the polymer is heated above the Ttrans, it returns to the primary shape. A thermal-mechanical cycle of the shape memory effect is complete.

In a 3D printing application using a bioplotter, the method of producing a 3D printed part with the flexible resorbable polymer, rigid resorbable particles, active pharmaceutical ingredients, or a mixture thereof includes either making a polymer solution with the said polymers or polymer pellets to be printed, or heated to melting point of said polymers. The polymer solution/melt is prepared and stored in a cartridge compatible with the 3D printer. The cartridge can be filled with polymer pellets for printing. A solid model is developed with the desired print geometry. The solid model is prepared for printing by performing a ‘slicing’ operation. The slicing operation separates the solid part geometry into the multiple layers that the printer is going to print. The layer height of the slices is determined by the operator and tip opening diameter. A petri dish mount can be secured to the platform. The petri dish used as a printing surface is placed within the mount. The prepared print geometry file is imported into the 3D printer software. The print is prepared by assigning a material to be used for the print and assigning a pattern to be used for the print infill. Additional factors are altered in this stage for the printing operation, but the two most basic changes are assigning a material to print with and a pattern for the print infill. A tip of desired diameter is added to the polymer solution cartridge and the cartridge is placed into the print head of the 3D printer. The print head containing the polymer solution is calibrated, and initial printing parameters are estimated and placed into the material profile in the 3D printer software. The printing operation is started by the operator. The printing head of the 3D printer moves in the x and y direction to print the part geometry. The print head then raises (z) and prints the next layer of the geometry. This process is repeated until the entire part has been printed.

In a 3D printing application using FFF, the method produces a 3D printed part with polymer material of flexible resorbable polymer, rigid resorbable particles, active pharmaceutical ingredients, or a mixture thereof. A solid model is developed with the desired print geometry. The solid model is prepared for printing by performing a ‘slicing’ operation. The slicing operation separates the solid part geometry into the multiple layers that the printer is going to print. The layer height of the slices is determined by the operator and tip opening diameter. The prepared print geometry file is imported into the 3D printer software. The print is prepared by assigning the polymer material to be used for the print and assigning a pattern to be used for the print infill. Polymer material feeds into the temperature-controlled FFF extrusion head, where it is heated to a semi-liquid state. The head extrudes and deposits the material in ultra-thin layers onto a fixtureless base. The head directs the material into place with precision. The material solidifies, laminating to the preceding layer. Parts are fabricated in layers, where each layer is built by extruding a small bead of material, or road, in a particular lay-down pattern, such that the layer is covered with the adjacent roads. After a layer is completed, the height of the extrusion head is increased and the subsequent layers are built to construct the part.

In a 3D printing application using binder jetting, the method produces a 3D printed part with polymer material powder of flexible resorbable polymer, rigid resorbable particles, active pharmaceutical ingredients, or a mixture thereof. A solid model is developed with the desired print geometry. The solid model is prepared for printing by performing a ‘slicing’ operation. The slicing operation separates the solid part geometry into the multiple layers that the printer is going to print. The layer height of the slices is determined by the operator and tip opening diameter. The prepared print geometry file is imported into the 3D printer software. The print is prepared by assigning the polymer material to be used for the print and assigning a pattern to be used for the print infill. The polymer materials powder is provided, then an amount of a binder is deposited onto the powder to produce an unfinished layer. The process is repeated to produce a three-dimensional unfinished model. The unfinished model is then sintered to produce a three-dimensional 3D printed part having a functionally-graded structure.

The preparation of the dog bone specimens were prepared according to ISO-527-1BB. The specimens shall be either directly injection- or compression-moulded from the material in accordance with ISO 293, ISO 294-1, ISO 295 or ISO 10724-1, as appropriate, or machined in accordance with ISO 2818 from plates that have been compression- or injection-moulded from the compound, or obtained from cast or extruded plates (sheet). The moulding conditions shall be in accordance with the relevant International Standard for the material or, if none exists, agreed between the interested parties. Strict control of all conditions of the specimen preparation is essential to ensure that all test specimens in a set are actually in the same state. All surfaces of the test specimen shall be free from visible flaws, scratches or other imperfections. From moulded specimens, all flash, if present, shall be removed, taking care not to damage the moulded surface. Test specimens from finished goods shall be taken from flat areas or zones having minimum curvature. For reinforced plastics, test specimens should not be machined to reduce their thickness unless absolutely necessary. Test specimens with machined surfaces will not give results comparable to specimens having nonmachined surfaces. The dimensions of the test specimens are as follows:

l3 Overall length ≥30
l1 Length of narrow parallel-sided portion 12.0±0.5

### r Radius ≥12

l2 Distance between broad parallel-sided portions 23±2
b2 Width at ends 4±02
b1 Width at narrow portion 2.0±0.2

### h Thickness ≥2

L0 Gauge length 10.0±0.2
L Initial distance between grips l2⁺¹0

The determination of the particle size, referred to diameter as well, in particular for the filler, was performed according to the United States Pharmacopeia 36 (USP) chapter <429> and European Pharmacopeia 7.0 (EP) chapter 2.9.31. The particle size distribution was determined utilizing a laser scattering instrument (e.g. Fa. Sympatec GmbH, type HELOS equipped with RODOS dry dispersing unit). The laser diffraction method is based on the phenomenon that particles scatter light in all directions with an intensity pattern that is dependent on particle size. A representative sample, dispersed at an adequate concentration in a suitable liquid or gas, is passed through the beam of a monochromic light source usually from a laser. The light scattered by the particles at various angles is measured by a multi-element detector, and numerical values relating to the scattering pattern are then recorded for subsequent analysis. Alternatively the diameter of the particles, e.g. filler, was determined via sieving.

### EXAMPLES

The following examples are put forth so as to provide those of ordinary skill in the art with a complete disclosure and description of how the polymer, particles, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Preparation of Example 1

### Processing of Polydioxanone With Starch

Polydioxanone (PDO, Evonik RESOMER® X 206 S commercially available from Evonik) with fillers, wherein the filler is biodegradable corn starch, with weight ratios between PDO and fillers of 100:0; 98:2; 95:5; 90:10 and 80:20 were compounded by a HAAKE MiniLab twin screw microcompounder. The 3 heating zones for the microcompounder were set at 140° C., respectively. PDO, or PDO with fillers were fed into the microcompounder and recirculated for 3 minutes and discharged to a HAAKE MiniJet cylinder at 145° C. The polymer melt was injection molded to dog-bone specimens following ISO-527-1BB. The melting and mold temperatures were 145° C. and 35° C., respectively. After melting, the material was injected into the mold with an injection pressure of 75 MPa for 8 seconds and a hold pressure of 45 MPa for 4 seconds.

### Preparation of Example 2

### Annealing of Specimens

Injection mold specimens of Example 1 were annealed under vacuum at 80° C. for 8 hours. To prevent thermal degradation during annealing after putting the specimens inside a vacuum oven, the chamber was degassed for 30 min at room temperature then heated to 80° C. and holding there for 8 hours.

### Preparation of Example 3

### Processing of PDO With PGA Particles

Polyglycolide (PGA, RESOMER® G 205 S commercially available from Evonik) was milled to particles. The PGA particles passed through sieves with diameter of 25 μm. Particle size was analyzed further by laser diffraction. PDO and PGA particles with weight ratios between PDO and PGA particles of 100:0; 95:5; 90:10 and 80:20 were compounded by a HAAKE MiniLab twin screw microcompounder. The 3 heating zones for the microcompounder were set at 140° C., respectively. PDO with PGA particles were fed into the microcompounder and recirculated for 3 minutes and discharged to a HAAKE MiniJet cylinder preheated at 145° C. The polymer melt was injection molded to dog-bone specimens following ISO-527-1BB. The melting and mold temperatures were 145° C. and 35° C., respectively. After melting, the material was injected into the mold with an injection pressure of 75 MPa for 8 seconds and a hold pressure of 45 MPa for 4 seconds.

### Testing

Mechanical properties including tensile strength, elastic modulus, elongation at break were measured on injection molded dog-bone shaped specimens (ISO-527-1BB) by an Instron Universal Testing Machine (Instron 3366) equipped with a 10 kN load cell and pneumatic grips (10 PSI air pressure). The specimens were then tested by tension mode with a crosshead speed of 5 mm/min or 20 mm/min at room temperature. Five replicates were tested, and average values were reported.

Shape memory property of the resorbable materials were evaluated by a Dynamic Mechanical Analyzer (DMA, Q-800, TA Instruments). Narrow section of injection molded specimens were cut to straight rectangular shape (18×2×1.5 mm), or cut from thermally compressive molded sheet to straight strip (20×3×0.3 mm) and mounted to the DMA with a pair of tensile clamps. The shape memory testing was performed by a controlled force method. The specimens were then heated to 40° C., held at 40° C. for 10 min, then applied 0.3-15 N force on the specimen. Afterwards, the specimens were cooled to −60° C. with applied constant force, held at −60° C. for 30 min, the force was unloaded and the specimen was heated to 40° C. and held at 40° C. for 30 min. Shape recovery rate is determined by Rr(%)=(εᵤ−εₚ)/(εₘ−εₚ)×100, where εᵤ, εₚ and εₘ represent the fixed strain after unloading, the permanent strain after heat-induced recovery, and the temporal strain achieved by deformation. All these strains were measured and recorded by the DMA.

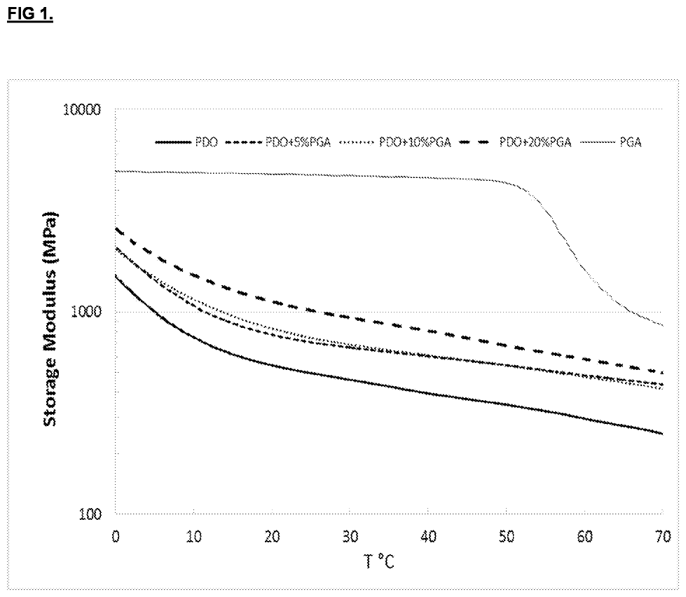

FIG. 1. depicts dynamic mechanical analysis of resorbable material containing PDO and PGA. The materials have improved storage modulus with inclusion of PGA particles. The dynamic mechanical properties of the materials with fillers was evaluated by a DMA (Q-800, TA Instruments). Narrow section of injection molded specimens were cut to straight rectangular shape (18×2×1.5 mm) and mounted it to the DMA with a pair of tensile clamps. The specimen was cooled to −60° C. then heated to 90° C. at a heating rate of 3° C./min. The specimens with more PGA particles showed a higher storage modulus. The PDO with 20% PGA particles have doubled storage modulus (840 MPa) compared to neat PDO (410 MPa) at 37° C.

Results

TABLE 1
Mechanical properties of materials consisting of resorbable
PDO and starch before (i.e., as-made) and after annealing.

| PDO/starch | Yield strength (MPa) | | Elongation at break (%) | | Elastic Modulus (MPa) | |
|---|---|---|---|---|---|---|
| ratio (w/w) | As-made | annealed | As-made | annealed | As-made | annealed |
| 100/0 | 27.7.8 ± 0.6 | 34.9 ± 1.0 | 215 ± 35 | 237 ± 61 | 593 ± 46 | 968 ± 56 |
| 98/2 | 24.7 ± 1.3 | 36.5 ± 0.4 | 266 ± 41 | 208 ± 51 | 810 ± 9 | 1010 ± 27 |
| 95/5 | 25.2 ± 2.1 | 36.1 ± 0.6 | 270 ± 27 | 164 ± 6 | 834 ± 30 | 1101 ± 44 |
| 90/10 | 27.4 ± 0.6 | 36.5 ± 0.4 | 246 ± 46 | 146 ± 28 | 973 ± 44 | 1205 ± 102 |
| 80/20 | 27.0 ± 2.2 | 31.7 ± 0.4 | 243 ± 5 | 140 ± 66 | 1065 ± 98 | 1328 ± 26 |

Inclusion of starch granules improve elastic modulus of PDO. Annealing as a post treatment process improves yield strength and elastic modulus of PDO and its composites with starch.

TABLE 2
Mechanical properties of materials consisting of resorbable
PDO and PGA before (i.e., as-made) and after annealing.

| PDO/PGA | Yield strength (MPa) | | Yield strain (%) | | Elastic Modulus (MPa) | |
|---|---|---|---|---|---|---|
| ratio (w/w) | As-made | annealed | As-made | annealed | As-made | annealed |
| 100/0 | 24.8 ± 0.9 | 34.9 ± 1.0 | 8.7 ± 0.5 | 8.8 ± 0.6 | 593 ± 46 | 968 ± 56 |
| 95/5 | 35.3 ± 0.1 | 39.33 ± 0.04 | 10.8 ± 0.1 | 11.0 ± 0.3 | 828 ± 50 | 849 ± 6 |
| 90/10 | 34.0 ± 0.1 | 38.7 ± 0.5 | 10.9 ± 0.3 | 11.2 ± 0.2 | 813 ± 14 | 910 ± 12 |
| 80/20 | 35.1 ± 0.6 | 39.2 ± 0.4 | 8.2 ± 0.2 | 8.2 ± 0.1 | 1030 ± 51 | 1009 ± 1 |

Inclusion of PGA particles and annealing process improve yield strength and elastic modulus of PDO making it mechanically strong and stiff. Yield strain is improved by inclusion of 5-10% PGA particles.

TABLE 3
Shape memory effect of the materials consisting of resorbable
PDO and starch granules or PGA particles.

| Composition | Shape recover rate (%) |
|---|---|
| PDO | 22 |
| PDO/20% starch | 50 |
| PDO/10% PGA | 43 |
| PDO/20% PGA | 70 |

The presence of rigid PGA particles or the starch granules enhances material's shape recover rate.
Item 1 is a resorbable material exhibiting a shape memory effect comprising a flexible resorbable polymer and at least one rigid resorbable filler.
Item 2 is the flexible resorbable polymer of item 1 is a semi-crystalline polymer.
Item 3 is the flexible resorbable polymer of item 1 is an amorphous polymer.
Item 4 is the flexible resorbable polymer of item 2 is a polydioxanone, a polycaprolactone, a poly(orthoester), a poly(phosphazene), a poly(hydroxybutyrate), a biodegradable polyurethane, a poly(amino acid), a polyetherester, polyphosphoesters, a polyanhydride, a multi-block copolymer of polydioxanone-b-polycaprolactone, a multi-block copolymer of poly(lactide-b-trimethylene carbonate), a multi-block copolymer of poly(glycolide-b-trimethylene carbonate), a multi-block copolymer of poly(lactide-b-caprolactone), a polyethylene glycol (PEG), a multi-block copolymer of polylactide-b-PEG, a multi-block copolymer of PGA-b-PEG, a multi-block copolymer of PCL-b-PEG, a multi-block copolymer of PDO-b-PEG, a multi-block copolymer of PEG and a polyorthoester, a multi-block copolymer of polyhydrobutyrate-b-polyhydroxyvalerate, and a copolymer, a terpolymer or a mixture thereof.
Item 5 is the flexible resorbable polymer of item 3 is a random copolymer of polydioxaone-co-polycaprolactone, a random copolymer of poly(lactide-co-caprolactone), a random copolymer of poly(lactide-co-trimethylene carbonate), a random copolymer of poly(glycolide-co-trimethylene carbonate), a random copolymer of polylactide-co-PEG, a random copolymer of PGA-co-PEG, a random copolymer of PCL-co-PEG, a random copolymer of PDO-co-PEG, a random copolymer of polyhydrobutyrate-co-polyhydroxyvalerate, or a mixture thereof.
Item 6 is the flexible resorbable polymer in item 1 is a mixture of one or more polymers in item 4 and one or more polymers in item 5.
Item 7 is the rigid resorbable filler of item 1 are in the form of powders, fines, granules, spheres, particles, crystalline whiskers, or a mixture thereof.
Item 8 is the rigid resorbable filler of item 7 have regular or irregular shape.
Item 9 is the rigid resorbable filler of item 7 have a diameter in the range of 0.01 to 100 μm, preferably in the range of 0.1 to 50 μm, and more preferably in the range of 0.1 to 20 or 25 μm.
Item 10 is the volume ratio between the flexible resorbable polymer and the rigid resorbable filler of item 1 is in the range of 99:1 to 50:50.

Item 11 is the rigid resorbable filler of item 7 is a polyglycolide, a polylactide, a poly (L-lactide-b-D,L-lactide), a polylactide-b-polyglycolide, a poly(L-lactide-co-D,L-lactide), a polylactide-co-polyglycolide, a polyesteramide, a polylactide stereocomplex, a starch granule, a cellulose microcrystal, a chitin whisker, a collagen, a crosslinked collagen, a silk, or a mixture thereof.

Item 12 is the flexible resorbable polymer of item 1 is a continuous matrix.
Item 13 is the rigid resorbable filler of item 1 is more rigid than the flexible resorbable polymer of item 1.
Item 14 is the resorbable material of item 1 further comprising an active pharmaceutical ingredient.
Item 15 is the active pharmaceutical ingredient in item 14 is dispersed in the flexible resorbable polymer, dispersed in the rigid resorbable filler, or dispersed in both the flexible resorbable polymer and rigid resorbable filler.
Item 16 is a process for preparing a material of item 1 by solvent mixing:

(a) dissolving the flexible resorbable polymer of item 1 in a solvent or a solvent mixture to make a solution;

(b) dispersing the rigid resorbable fillers in the solution;

(c) removing the solvents; and

(d) forming the material.

Item 17 is a thermal process for preparing a material of item 1 by extrusion:

(a) feeding a flexible resorbable polymer, and rigid resorbable filler(s) to an extruder;

(b) compounding the flexible resorbable polymer and rigid resorbable fillers using the extruder at a temperature above the melting temperature of flexible resorbable polymer;

(c) extruding the materials; and

(d) forming the materials into a shape using a die.

Item 18 is a thermal process for preparing a material of item 1 by injection molding:

(a) feeding a flexible resorbable polymer, and rigid resorbable filler(s) to an injection molding machine;

(b) melting the flexible resorbable polymer above the melting temperature of flexible resorbable polymer but below the melting temperature the rigid resorbable fillers;

(c) injecting the materials into a mold cavity; and

(d) forming the materials into a shape using a mold.

Item 19 is a thermal process for preparing a material of item 1 by thermoforming:

(a) placing sheet(s) of a material of item 1 in a mold;

(b) heating the sheet(s) to pliable, and

(c) forming the sheet(s) into a shape.

Item 20 is a process for producing a 3D printed part from a material of item 1 using a bioplotter; the process comprising:

(a) feeding a flexible resorbable polymer, and rigid resorbable filler(s) to a cartridge;

(b) melting the flexible resorbable polymer above the melting temperature of flexible resorbable polymer but below the melting temperature the rigid resorbable fillers;

(c) printing the material through a print head to form multiple layers of the 3D printed part; and

(d) setting the 3D printed part.

Item 21 is a process for producing a 3D printed part from a material of item 1 using binder jetting, comprising the steps of:

(a) providing powders of material of item 1;

(b) selectively depositing an amount of a binder onto the powders of material to produce an unfinished layer;

(c) repeating steps (a) and (b) to produce a three-dimensional unfinished model; and

(d) sintering the unfinished model to produce a three-dimensional 3D printed part.

Item 22 is a process for producing a 3D printed part from a material of item 1 using FFF, comprising the steps of:

(a) feeding the filament of the material of item 1 into a temperature-controlled FFF extrusion head;

(b) heating the extrusion head for the materials of item 1 to form a semi-liquid state material;

(c) extruding the material;

(d) depositing the material onto a fixtureless base; wherein the head directs the material into place forming thin layers of the material, and

(e) solidify the material by laminating the material to the preceding layer to form a 3D printed part.

Item 23 is a process for producing a 3D printed part from a material of item 1 using SLS, comprising the steps of:

(a) dispersing a thin layer on top of a platform inside the build chamber;

(b) the laser scans a cross-section of the 3D model and heats the powder around the melting point of the material;

(c) the platform lowers by one layer into the build chamber, and redispersing a new thin layer of the powder on top. The laser scans the next cross-section of the build, and

(d) repeating the steps from (a) to (c) to form a 3D printed part.

Item 24 is the material comprising the flexible resorbable polymer of item 1 and rigid resorbable fillers having improved rigidity than the flexible resorbable polymer of item 1.
Item 25 is the material comprising the flexible resorbable polymer of item 1 and rigid resorbable fillers of item 1 is resorbable.
Item 26 is the material comprising the flexible resorbable polymer of item 2 and rigid resorbable filler of item 1 has higher crystallinity than the flexible resorbable polymer of item 2.
Item 27 is the resorbable material in item 1 has improved mechanical strength than the flexible resorbable polymer of item 1.
Item 28 is the resorbable material in item 1 has improved yield strain than the flexible resorbable polymer of item 1.
Item 29 is the resorbable material in item 1 has improved yield strength than the flexible resorbable polymer of item 1.
Item 30 is the material of item 1 can be used for bone filler, vascular closure and other hemostasis devices, aneurysms, stent, fast degradation applications, drug delivery, and drug release applications, or any other medical application requiring implanting into the human body.

What is claimed is:

1. A resorbable material exhibiting a shape memory effect comprising a flexible resorbable polymer and at least one rigid resorbable filler.

2. The resorbable material of claim 1, wherein the flexible resorbable polymer
i) is a semi-crystalline polymer; or
ii) is an amorphous polymer; or
iii) is a semi-crystalline polymer selected from a polydioxanone, a polycaprolactone, a poly(orthoester), a poly(phosphazene), a poly(hydroxybutyrate), a biodegradable polyurethane, a poly(amino acid), a polyetherester, polyphosphoesters, a polyanhydride, a multi-block copolymer of polydioxanone-b-polycaprolactone, a multi-block copolymer of poly(lactide-b-trimethylene carbonate), a multi-block copolymer of poly(glycolide-b-trimethylene carbonate), a multi-block copolymer of poly(lactide-b-caprolactone), a polyethylene glycol (PEG), a multi-block copolymer of polylactide-b-PEG, a multi-block copolymer of PGA-b-PEG, a multi-block copolymer of PCL-b-PEG, a multi-block copolymer of PDO-b-PEG, a multi-block copolymer of PEG and a polyorthoester, a multi-block copolymer of polyhydrobutyrate-b-polyhydroxyvalerate, and a copolymer, a terpolymer or a mixture thereof; or
iv) is an amorphous polymer selected from a random copolymer of polydioxaone-co-polycaprolactone, a random copolymer of poly(lactide-co-caprolactone), a random copolymer of poly(lactide-co-trimethylene carbonate), a random copolymer of poly(glycolide-co-trimethylene carbonate), a random copolymer of polylactide-co-PEG, a random copolymer of PGA-co-PEG, a random copolymer of PCL-co-PEG, a random copolymer of PDO-co-PEG, a random copolymer of polyhydrobutyrate-co-polyhydroxyvalerate, or a mixture thereof; or
v) is a mixture of at least one semi-crystalline polymer selected from a polydioxanone, a polycaprolactone, a poly(orthoester), a poly(phosphazene), a poly(hydroxybutyrate), a biodegradable polyurethane, a poly(amino acid), a polyetherester, polyphosphoesters, a polyanhydride, a multi-block copolymer of polydioxanone-b-polycaprolactone, a multi-block copolymer of poly(lactide-b-trimethylene carbonate), a multi-block copolymer of poly(glycolide-b-trimethylene carbonate), a multi-block copolymer of poly(lactide-b-caprolactone), a polyethylene glycol (PEG), a multi-block copolymer of polylactide-b-PEG, a multi-block copolymer of PGA-b-PEG, a multi-block copolymer of PCL-b-PEG, a multi-block copolymer of PDO-b-PEG, a multi-block copolymer of PEG and a polyorthoester, a multi-block copolymer of polyhydrobutyrate-b-polyhydroxyvalerate and at least one amorphous polymer selected from a random copolymer of polydioxaone-co-polycaprolactone, a random copolymer of poly(lactide-co-caprolactone), a random copolymer of poly(lactide-co-trimethylene carbonate), a random copolymer of poly(glycolide-co-trimethylene carbonate), a random copolymer of polylactide-co-PEG, a random copolymer of PGA-co-PEG, a random copolymer of PCL-co-PEG, a random copolymer of PDO-co-PEG, a random copolymer of polyhydrobutyrate-co-polyhydroxyvalerate; and/or
vi) is in the form of a continuous matrix.

3. The resorbable material of claim 1 or 2, wherein the rigid resorbable filler
i) are in the form of powders, fines, granules, spheres, particles, crystalline whiskers, or a mixture thereof; and/or
ii) have regular or irregular shape; and/or
iii) have a diameter in the range of 0.01 to 100 μm, preferably in the range of 0.1 to 50 μm, and more preferably in the range of 0.1 to 25 μm; and/or
iv) are a polyglycolide, a polylactide, a poly (L-lactide-b-D,L-lactide), a polylactide-b-polyglycolide, a poly(L-lactide-co-D,L-lactide), a polylactide-co-polyglycolide, a polyesteramide, a polylactide stereocomplex, a starch granule, a cellulose microcrystal, a chitin whisker, a collagen, a crosslinked collagen, a silk, or a mixture thereof; and/or
v) are more rigid than the flexible resorbable polymer.

4. The resorbable material according to any of claims 1 to 3, wherein
i) the volume ratio between the flexible resorbable polymer and the rigid resorbable filler is in the range of 99:1 to 50:50; and/or
ii) the resorbable material has improved rigidity compared to the flexible resorbable polymer; and/or
iii) the resorbable material has higher crystallinity than the flexible resorbable polymer; and/or
iv) the resorbable material has improved mechanical strength than the flexible resorbable polymer; and/or
v) the resorbable material has improved yield strain compared to the flexible resorbable polymer.

5. The resorbable material of any of claims 1 to 4 further comprising an active pharmaceutical ingredient.

6. The resorbable material of claim 5, wherein the active pharmaceutical ingredients are dispersed in the flexible resorbable polymer, dispersed in the rigid resorbable filler, or dispersed in both the flexible resorbable polymer and rigid resorbable filler.

7. A process for preparing a resorbable material of any of claims 1 to 6 by solvent mixing, comprising the steps:
(a) dissolving the flexible resorbable polymer in a solvent or a solvent mixture to make a solution;
(b) dispersing the rigid resorbable fillers in the solution;
(c) removing the solvents; and
(d) forming the material.

8. A thermal process for preparing a resorbable material of any of claims 1 to 6 by extrusion, comprising the steps:
(a) feeding a flexible resorbable polymer, and rigid resorbable filler(s) to an extruder;
(b) compounding the flexible resorbable polymer and rigid resorbable fillers using the extruder at a temperature above the melting temperature of the flexible resorbable polymer to form a mixture;
(c) extruding the mixture; and
(d) forming the mixture into a shape using a die.

9. A thermal process for preparing a resorbable material of any of claims 1 to 6 by injection molding, comprising the steps:
(a) feeding a flexible resorbable polymer, and rigid resorbable filler(s) to an injection molding machine;
(b) melting the flexible resorbable polymer above the melting temperature of flexible resorbable polymer but below the melting temperature the rigid resorbable fillers to form a mixture;
(c) injecting the mixture into a mold cavity; and
(d) forming the mixture into a shape using a mold.

10. A thermal process for preparing a resorbable material of any of claims 1 to 6 by thermoforming, comprising the steps:
(a) placing sheet(s) of the resorbable material in a mold;
(b) heating the sheet(s) to pliable, and
(c) forming the sheet(s) into a shape.

11. A process for producing a 3D printed part from a material of any of claims 1 to 6 using a bioplotter; the process comprising:
(a) feeding a flexible resorbable polymer, and rigid resorbable filler(s) to a cartridge;
(b) melting the flexible resorbable polymer above the melting temperature of flexible resorbable polymer but below the melting temperature of the rigid resorbable fillers to obtain a mixture;
(c) printing the mixture through a print head to form multiple layers of the 3D printed part; and
(d) setting the 3D printed part.

12. A process for producing a 3D printed part from a resorbable material of any of claims 1 to 6 using binder jetting, comprising the steps of:
(a) providing powders of the resorbable material;
(b) selectively depositing an amount of a binder onto the powders of material to produce an unfinished layer;
(c) repeating steps (a) and (b) to produce a three-dimensional unfinished model; and
(d) sintering the unfinished model to produce a three-dimensional 3D printed part.

13. A process for producing a 3D printed part from a resorbable material of any of claims 1 to 6 using FFF, comprising the steps of:
(a) feeding the filament of the resorbable material into a temperature-controlled FFF extrusion head;
(b) heating the extrusion head for the resorbable material to form a semi-liquid state resorbable material;
(c) extruding the resorbable material;
(d) depositing the extruded resorbable material onto a fixtureless base; wherein the head directs the material into place forming thin layers of the extruded resorbable material, and
(e) solidify the extruded resorbable material by laminating the material to the preceding layer to form a 3D printed part.

14. A process for producing a 3D printed part from a resorbable material of any of claims 1 to 6 using SLS, comprising the steps of:
(a) dispersing a thin layer on top of a platform inside the build chamber;
(b) the laser scans a cross-section of the 3D model and heats the powder around the melting point of the resorbable material;
(c) the platform lowers by one layer into the build chamber, and redispersing a new thin layer of the powder on top, the laser scans the next cross-section of the build, and
(d) repeating the steps from (a) to (c) to form a 3D printed part.

15. 3D printed part obtained by a process according to any of claims 11 to 14.

16. Use of the resorbable material according to any of claims 1 to 6 or the 3D printed part of claim 15 as bone filler, vascular closure and other hemostasis devices, aneurysms, stent, fast degradation applications, drug delivery, and drug release applications, or any other medical application requiring implanting into the human body.
